(19) United States
(12) Patent Application Publication
(10) Pub. No.: US 2009/0054533 A1
(43) Pub. Date: Feb. 26, 2009

(54) Film coating compositions based on tribasic calcium phosphate

(76) Inventors: Suresh Rukmanand Pareek, Mumbai (IN); Chetan Rajsharad, Dist-Thane (IN)

Correspondence Address: BLANK ROME LLP, 600 NEW HAMPSHIRE AVENUE, N.W., WASHINGTON, DC, 20037, US

(21) Appl. No.: 12/073,731

(22) Filed: Mar. 10, 2008

(30) Foreign Application Priority Data
Aug. 20, 2007 (IN) 1596/MUM/2007

(51) Int. Cl. C08K 3/32; A61K 47/02; C08K 5/3415; C08K 5/15; C08K 5/20; A01C 1/06; A23P 1/08; A23J 7/00; A23L 1/27; A23L 1/236; A23L 1/22

(52) U.S. Cl. 514/769; 524/414; 524/95; 524/56; 524/217; 524/58; 427/4; 426/531; 426/662; 426/540; 426/548; 426/650; 426/302

(57) ABSTRACT

Disclosed herein are dry film coating compositions comprising of tribasic calcium phosphate as one of the essential ingredient along with other commonly used polymers, plasticizers, excipients like suspending agents, pigments, lubricants, glidents, opacifiers, flavours, sweetener etc. for use in coating of pharmaceutical tablets, nutritional supplements, food products, confectionery, agricultural seeds and the like The invention further discloses method for coating a substrate using the invented coating composition.

### TECHNICAL FIELD

This invention relates to the field of coating compositions for substrates like pharmaceutical tablets, nutritional supplements, food products, confectionery, agricultural seeds for providing protection to the substrate from environmental conditions like light, humidity, atmospheric gases etc.

### BACKGROUND OF THE INVENTION

Film coating is generally, a process of depositing a thin layer of material onto a substrate such as a pharmaceutical tablet to provide a protective barrier covering the outer surface and also to provide an aesthetic appearance to the product.

There is ample literature in the form of patents and published articles available on film coating and its compositions. There are various types of film coating compositions known in the art and have a wide range of applications in coating like pharmaceutical tablets, food products, agricultural seeds etc. Most of these compositions use certain polymers like HPMC, HPC, HEC, and other cellulosic polymers.

Japanese patent JP H8-59512 provides compositions for film coating based on polyvinyl alcohol as film forming polymers. U.S. Pat. No. 3,935,326 filed in April 1974 describes the coating of core tablets with polymers selected from the group consisting of polyvinyl alcohol, polyvinyl pyrrolidone and solid polyethylene glycol. Subsequent to these patents, U.S. Pat. No. 5,885,617 filed in June 1995 having the application priority of GB9414045 (Jul. 12, 1994) provides moisture barrier film coating compositions based on polyvinyl alcohol. Yet another U.S. Pat. No. 5,604,282 filed in December 1994 provides a strippable, rewettable film coating generally comprising polyvinyl polymer, polyvinyl alcohol and other ingredients.

However, all these compositions were found to be fairly sticky in nature, making the film coating process quite difficult. Some of these difficulties have been claimed to be resolved in the film coating compositions described in U.S. Pat. No. 6,448,323 filed in July 1999.

In addition to the above compositions based on polyvinyl alcohol, there have been many compositions known in the art of film coating based on various cellulosic polymers.

All these polymers which have been used for film coating provide fairly tacky coating suspensions which when sprayed on the substrates like pharmaceutical tablets; form a thin film on the substrate. Inventors have tried to add lubricants like talc and other inert excipients like pigments/opacifiers etc to reduce the tackiness of the polymers and to increase the solid content thereby reducing the time of coating. However, the problem of tackiness still persists.

Even though various types of film coating compositions are known in the art and are available to the industry for wide range of applications; many of such compositions use expensive ingredients which may have restricted supplies or availability.

Therefore, there remains a long felt need in the art to develop cost-effective film coating compositions that eliminates the above mentioned drawbacks such as tackiness, low tensile strength and low spray rate etc. This forms the subject of the present invention.

### OBJECT OF THE INVENTION

The objective of the invention is to provide dry film coating composition that has a smooth surface, shiny gloss, minimal tackiness, good film strength and adhesion properties and provides excellent protection to the substrate against various environmental conditions like light, moisture and atmospheric gases.

Another objective of the invention is to provide cost-effective film coating compositions thereby making this composition available to a wide range of food and medicinal products.

Further objective of the invention is to provide a method for coating the substrates using the composition of the invention.

### SUMMARY OF THE INVENTION

In accordance with the above objectives, the present invention provides cost-effective dry film coating compositions comprising of tribasic calcium phosphate as one of the essential ingredient along with other commonly used polymers, plasticizers, excipients like suspending agents, pigments, lubricants, glidents, opacifiers, flavours, sweetener etc. for use in coating of pharmaceutical tablets, nutritional supplements, food products, confectionery, agricultural seeds and the like.

In another aspect, the invention provides a method of coating a substrate using the invented coating composition.

### DETAILED DESCRIPTION

The invention will now be described in detail in connection with certain preferred and optional embodiments, so that various aspects thereof may be more fully understood and appreciated.

It is well known in the art of film coating that polyvinyl alcohol is a very tacky polymer which makes the coating process very difficult for the coating compositions where polyvinyl alcohol has been used as the main film forming polymer.

Thus the present inventors, on exploring various alternatives for a reliable film coating compositions, have been found that the use of an inert pharmaceutical excipient, tribasic calcium phosphate in film coating compositions reduces the tackiness of polyvinyl alcohol very significantly, thus makes the film coating process much simpler and easy. Even after reducing the polymer tackiness, use of tribasic calcium phosphate still provides good film strength and high film adhesion so that the resulting film provides much improved protection to the substrate.

The present invention describes dry film coating compositions wherein an inexpensive and commonly used pharmaceutical excipient, tribasic calcium phosphate has been used along with other commonly used polymers to achieve a low cost coating composition.

Accordingly, dry film coating compositions of the present invention suitable for use in coating pharmaceutical tablets, nutritional supplements, food, confectionery or agricultural seeds comprises tribasic calcium phosphate in combination with polymer system and plasticizer. The invented composition has excellent film forming ability and film adhesion to the substrate. It has also been observed that the invented composition provides excellent protection to the substrate against various environmental conditions like light, moisture and atmospheric gases.

Advantageously, the tribasic calcium phosphate can be used in the composition in a range of about 2% to about 50% more preferably in a range of about 6% to about 40% by weight of the composition.

The present inventors have further experimented the compatibility of tribasic calcium phosphate with other polymers like HPMC, HPC, HEC, methyl cellulose, ethyl cellulose, aqueous dispersion of ethyl cellulose, sodium alginate, alginic acid, sodium carboxy methyl cellulose and found that tribasic calcium phosphate is an excellent excipient and can easily be used with other polymers also with superior results. Thus, the polymer system of present inventive formulation may comprise one or more polymers as mentioned above. The polymer system of present invention may present in a range of 10% to 90%; more preferably in a range of about 15% to about 75% by weight of the composition. The film adhesion was found to be higher when the polymer was mixed with tribasic calcium phosphate than when used alone. The use of tribasic calcium phosphate does not increase the viscosity of the coating suspension, hence, the solid content of the coating suspension can also be increased than using the polymer alone thus, making the coating process quite faster. It was further observed that in certain compositions based on tribasic calcium phosphate, the solid content of the coating suspension can be increased upto 25% w/w without having any adverse effect on spray characteristics.

Tribasic calcium phosphate was found to be an excellent excipient for film coating as it is compatible with a wide variety of plasticizers without affecting the shelf life of the product and hence the composition may advantageously contain one or more plasticizers. Various plasticizers which have been used in different compositions are triacetin, propylene glycol, various grades of polyethylene glycol, liquid paraffin, diethyl phthalate, dibutyl phthalate, triethyl citrate, lecithin, fractionated coconut oil, castor oil etc. in different concentrations ranging from 2% to 30% w/w. The more preferable range is about 5% to about 20% by weight of the composition.

Further, the inventors have also found that the addition of liquid plasticizers like PEG 200 or PEG 400 or triacetin or castor oil or diethyl phthalate or dibutyl phthalate etc. is much easier in the coating compositions based on tribasic calcium phosphate. The liquid plasticizers get adsorbed onto tribasic calcium phosphate and does not make the whole composition wet. This nature of tribasic calcium phosphate helps in adding higher concentrations of liquid plasticizers (which is not possible in many known compositions in art) and also makes the manufacturing of coating composition much easier.

The composition of the present invention further includes a pigment/opacifier in an amount of 0% to 30% by weight of the composition.

Tribasic calcium phosphate is proved to be a useful excipient in film coating compositions as it was found to be compatible with other commonly used excipients in film coating like talc, fumed silica, kaolin, magnesium stearate, stearic acid, opacifiers like titanium dioxide and calcium carbonate, colours (synthetic dyes, natural colours, iron oxide colours as well as candurin colours), flavours, sweeteners etc. The product performance and shelf life was found to be good when coating composition has tribasic calcium phosphate as one of the ingredient.

The invented composition further comprises flow aid selected from talc, fumed silica, kaolin, magnesium stearate, stearic acid or magnesium trisilicate, either alone or in combination of more than one ingredient. The flow aid is present in an amount ranging 0% to 20% by weight of the composition.

The dry film coating composition further comprises colouring agent selected from lake colours, soluble colours, iron oxide colours, candurin colours or natural colours either alone or a combination thereof. The amount of colouring agent may present in the range of 0% to 30% by weight of the composition.

A sweetener selected from sodium saccharin, sucrose, aspartame, neutrame or dextrose, may also present in the film coating composition either alone or in mixtures thereof. The sweetener may present in a range of about 0% to about 10% by weight of the composition.

The film coating composition may further contain a flavouring agent in an amount ranging from 0% to 15% by weight of the composition. The flavouring agent being either encapsulated powder or liquid flavour suitable for pharmaceutical/food applications can be used.

Advantageously, the presence of tribasic calcium phosphate in film coating composition makes the product less hygroscopic and thus reduces the caking tendency of various polymers; many of those tend to pick moisture from atmosphere in high humidity conditions and tend to cake. Thus, the presence of tribasic calcium phosphate helps in increased shelf life of the coating composition.

It has been further observed that commercially available grades of tribasic calcium phosphate give satisfactory to good results, but the performance of the coating composition improved significantly once the micronized grade of tribasic calcium phosphate was used. The surface coverage by the resulting film, film smoothness and gloss level were comparatively better with the micronized grade of tribasic calcium phosphate. Therefore, micronized tribasic calcium phosphate with a micron size of 10 to 200 microns can be used advantageously to achieve the desired results.

The invented composition can very easily be manufactured by dry mixing of various ingredients (mixing may need specific order of addition depending on the ingredients used), carrying out the colour matching exercise using spectrophotometric colour scan method.

In another preferred embodiment, the invention describes a method for coating substrates. Accordingly, a method for coating substrates such as pharmaceutical tablets, nutritional supplements, food, confectionery, agricultural seeds, and the like, with a film coating composition comprising the steps of:
a) mixing the coating composition into water or into organic solvents or into a combination of water and alcoholic solvents to form a smooth dispersion;
b) applying an effective amount of said coating dispersion onto said substrate to form a film on the said substrate, and
c) drying the film coating on said substrate.

In accordance with the above method, the dry mix composition can easily be converted into coating suspension by suspending in suitable solvent system using a simple stirring procedure. The suspension becomes ready for coating quite quickly when a propeller stirrer is used for suspension preparation.

The invented composition can easily be mixed with various solvent systems like purified water, a mixture of purified water and alcoholic solvents (like ethyl alcohol, isopropyl alcohol, methyl alcohol etc.), a mixture of alcoholic solvents and other organic solvents like methylene dichloride, acetone etc. The use of solvent system was found to be mostly governed by the use of polymer/polymer system and the nature of the substrate to be coated.

The invented composition can easily work in fairly wide range of film coating process conditions which can easily be controlled, monitored and reproduced. Once the suspension is ready, it can easily be sprayed onto the substrate using commonly used spray equipments and the coating process can easily be carried out either in conventional (non perforated) coating pan, perforated coating pan (like Acela Cota) or in fluid bed coater.

Thus, the invention covers the novel film coating compositions and includes coating dispersions, the coated substrates, such as coated pharmaceutical tablets, coated nutritional supplements, coated food products, coated confectionery forms, coated agricultural seeds etc and the method of making the dry film coating compositions and the method of making the coating dispersion of the invention.

The following examples, which include preferred embodiments, will serve to illustrate the practice of this invention, it being understood that the particulars shown are by way of example and for purpose of illustrative discussion of preferred embodiments of the invention.

The ranges of each component of the dry coating compositions of the invention are as follows, by weight:

| Component | Acceptable ranges (%) | Preferred ranges (%) |
|---|---|---|
| Tribasic calcium phosphate | 2-50 | 6-40 |
| Polymer | 10-90 | 15-75 |
| Plasticizer | 2-30 | 5-20 |
| Talc | 0-8 | 0-5 |
| Pigment/Opacifier/Colour | 0-35 | 0-30 |

The following examples illustrate the practice of invention. All units and percentages in the examples are given by weight.

### EXAMPLE 1

General method for preparation of the coating composition and coating process:

The dry components of the following formulation were blended together in a dry powder mill or a blender, for 30 minutes, until an homogenous mixture achieved, resulting in the formulation of the inventive dry film coating composition. Optionally, the dry film coating composition of the invention may be granulated using a planetary mixer. After the dry film coating composition was loaded into the mixer, sufficient water was slowly added during the mixing process until the composition forms slightly adherent granules. These granules were then passed through a 1-2 mm screen and then dried in an oven at 30° C. until the moisture content below 5%. The composition was then sieved again through a 1-2 mm screen to make it ready for use in a nondusting, granular form.

The composition may optionally be milled such as in a hammer mill, for example. Other methods of granulation, which may also be used, are spray granulation and roller compaction.

100 grams of the resulting film coating composition was dispersed into 400 grams of distilled water to form an inventive coating dispersion (20% solids), and 500 grams of the above dispersion was sprayed onto 3 kilograms of tablets using a coater to form the inventive coating on the tablets having a theoretical weight gain of 3%. The tablets were rotated in the coater at a rotation speed of 12 rpm. Hot air at 60° C. to 65° C., was used to evaporate the solvent in the sprayed coating dispersion and to maintain the tablets at around 38° C. The spraying rate of coating dispersion during coating was 15 grams per minute.

Upon completion of the coating process, the tablets showed a smooth surface with excellent logo definition. The film coating on the tablets possesses an excellent long-lasting shinny gloss, minimal tackiness, good film adhesion, and good tensile strength.

| Component | Percentages | Grams |
|---|---|---|
| Tribasic calcium phosphate | 40 | 400 |
| Polyvinyl alcohol | 44 | 440 |
| Polyethylene glycol 6000 | 16 | 160 |
| Total | 100% | 1000 |

### EXAMPLE 2

| Component | Percentages | Grams |
|---|---|---|
| Tribasic calcium phosphate | 20 | 200 |
| Polyvinyl alcohol | 44 | 440 |
| Polyethylene glycol 4000 | 16 | 160 |
| Titanium dioxide | 12 | 120 |
| Lake colour | 8 | 80 |
| Total | 100% | 1000 |

### EXAMPLE 3

| Component | Percentages | Grams |
|---|---|---|
| Tribasic calcium phosphate | 19 | 190 |
| Polyvinyl alcohol | 38 | 380 |
| Polyethylene glycol 6000 | 8 | 80 |
| Polyethylene glycol 400 | 5 | 50 |
| Talc | 5 | 50 |
| Titanium dioxide | 15 | 150 |
| Iron oxide yellow | 10 | 100 |
| Total | 100% | 1000 |

### EXAMPLE 4

| Component | Percentages | Grams |
|---|---|---|
| Tribasic calcium phosphate | 15 | 150 |
| Polyvinyl alcohol | 56 | 560 |
| Polyethylene glycol 4000 | 13 | 130 |
| Talc | 5 | 50 |
| Iron oxide red | 11 | 110 |
| Total | 100% | 1000 |

### EXAMPLE 5

| Component | Percentages | Grams |
|---|---|---|
| Tribasic calcium phosphate | 40 | 400 |
| Polyvinyl alcohol | 15 | 150 |
| Polyethylene glycol 8000 | 17 | 170 |
| Polyethylene glycol 400 | 8 | 80 |
| Titanium dioxide | 10 | 100 |
| Lake colour | 10 | 100 |
| Total | 100% | 1000 |

### EXAMPLE 6

| Component | Percentages | Grams |
|---|---|---|
| Tribasic calcium phosphate | 15 | 150 |
| HPMC | 60 | 600 |
| Polyethylene glycol 6000 | 15 | 150 |
| Iron oxide yellow | 10 | 100 |
| Total | 100% | 1000 |

### EXAMPLE 7

| Component | Percentages | Grams |
|---|---|---|
| Tribasic calcium phosphate | 12 | 120 |
| HPMC | 40 | 400 |
| HPC | 20 | 200 |
| Triacetin | 8 | 80 |
| Talc | 4 | 40 |
| Titanium dioxide | 11 | 110 |
| Lake Colour | 5 | 50 |
| Total | 100% | 1000 |

### EXAMPLE 8

| Component | Percentages | Grams |
|---|---|---|
| Tribasic calcium phosphate | 18 | 180 |
| HPMC | 56 | 560 |
| Ethyl cellulose (aq. dispersion) | 6 (total solids) | 60 |
| Triacetin | 8 | 80 |
| Titanium dioxide | 8 | 80 |
| Lake Colour | 4 | 40 |
| Total | 100% | 1000 |

### EXAMPLE 9

| Component | Percentages | Grams |
|---|---|---|
| Tribasic calcium phosphate | 10 | 100 |
| HPMC | 55 | 550 |
| Ethyl cellulose | 5 | 50 |
| Diethyl phthalate | 10 | 100 |
| Talc | 7 | 70 |
| Titanium dioxide | 10 | 100 |
| Iron oxide red | 3 | 30 |
| Total | 100% | 1000 |

### EXAMPLE 10

| Component | Percentages | Grams |
|---|---|---|
| Tribasic calcium phosphate | 30 | 300 |
| HPMC | 30 | 300 |
| Polyvinyl alcohol | 15 | 150 |
| Polyethylene glycol 400 | 10 | 100 |
| Polyethylene glycol 6000 | 4 | 40 |
| Iron oxide yellow | 9 | 90 |
| Lake Colour | 2 | 20 |
| Total | 100% | 1000 |

### EXAMPLE 11

| Component | Percentages | Grams |
|---|---|---|
| Tribasic calcium phosphate | 8 | 80 |
| HPMC | 60 | 600 |
| Polyethylene glycol 6000 | 12 | 120 |
| Titanium dioxide | 3 | 30 |
| Lake Colour | 8 | 80 |
| Flavour | 8.5 | 85 |
| Sodium saccharin | 0.5 | 5 |
| Total | 100% | 1000 |

### EXAMPLE 12

| Component | Percentages | Grams |
|---|---|---|
| Tribasic calcium phosphate | 8 | 80 |
| Polyvinyl alcohol | 44 | 440 |
| Polyethylene glycol 6000 | 14 | 140 |
| Talc | 5 | 50 |
| Iron oxide red | 20 | 200 |
| Flavour | 8 | 80 |
| Aspartame | 1 | 10 |
| Total | 100% | 1000 |

### EXAMPLE 13

| Component | Percentages | Grams |
|---|---|---|
| Tribasic calcium phosphate | 20 | 200 |
| Polyvinyl alcohol | 40 | 400 |
| Polyethylene glycol 6000 | 12 | 120 |
| Soya Lecithin | 3 | 30 |
| Titanium dioxide | 7 | 70 |
| Candurin colour | 18 | 180 |
| Total | 100% | 1000 |

### EXAMPLE 14

| Component | Percentages | Grams |
|---|---|---|
| Tribasic calcium phosphate | 15 | 150 |
| HPMC | 56 | 560 |
| Polyethylene glycol 6000 | 12 | 120 |
| Talc | 4 | 40 |
| Titanium dioxide | 5 | 50 |
| Candurin colour | 8 | 80 |
| Total | 100% | 1000 |

### EXAMPLE 15

| Component | Percentages | Grams |
|---|---|---|
| Tribasic calcium phosphate | 20 | 200 |
| Polyvinyl alcohol | 32 | 320 |
| Soya lecithin | 4 | 40 |
| Polyethylene glycol 8000 | 15 | 150 |
| Talc | 5 | 50 |
| Titanium dioxide | 20 | 200 |
| Lake Colour | 4 | 40 |
| Total | 100% | 1000 |

The coating formulations as described above (examples 2 to 15) are prepared similarly in accordance with the above method exemplified under example 1.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1. A dry film coating composition for use in coating pharmaceutical tablets, nutritional supplements, food, confectionery or agricultural seeds, said film coating composition comprising: tribasic calcium phosphate present in the range of about 2% to about 50% by weight of the composition; a polymer systems; and a plasticizer.

2. The coating composition as claimed in claim 1, wherein said tribasic calcium phosphate used is micronized to a micron size of 10 to 200 microns.

3. The coating composition as claimed in claim 1, wherein said tribasic calcium phosphate is present in a range of about 6% to about 40% by weight of the composition.

4. The coating composition as claimed in claim 1, wherein the polymer used in polymer system is selected from the group consisting of hydroxypropyl methyl cellulose, hydroxy ethyl cellulose, hydroxy propyl cellulose, methyl cellulose, ethyl cellulose, aqueous dispersion of ethyl cellulose, polyvinyl alcohol, sodium alginate, alginic acid, sodium carboxy methyl cellulose, and combinations thereof.

5. The coating composition as claimed in claim 1, wherein said polymer system is polyvinyl alcohol.

6. The coating composition as claimed in claim 5, wherein said polymer system is present in a range of about 10% to about 90%.

7. The coating composition as claimed in claim 1, wherein said plasticizer is selected from the group consisting of polyethylene glycol, propylene glycol, glycerin, triacetin, diethyl phthalate, dibutyl phthalate, lecithin, liquid paraffin, triethyl citrate, fractionated coconut oil, castor oil, and combinations thereof.

8. The coating composition as claimed in claim 1, wherein said plasticizer is polyethylene glycol.

9. The coating composition as claimed in claim 7, wherein said plasticizer is present in a range of about 2% to about 30%.

10. The coating composition as claimed in claim 1, further comprising a pigment/opacifier.

11. The coating composition as claimed in claim 10, wherein said pigment/opacifier is present in a range of 0% to about 30% by weight of the composition.

12. The coating composition as claimed in claim 1, further comprising a flow aid selected from the group consisting of talc, fumed silica, kaolin, magnesium stearate, stearic acid, magnesium trisilicate, and combinations thereof.

13. The coating composition of claim 12, wherein the flow aid is present in a range of about 0% to about 20% by weight of the composition.

14. The coating composition as claimed in claim 1, further comprising a colouring agent selected from the group consisting of lake colours, soluble colours, iron oxide colours, candurin colours, natural colours, and combinations thereof.

15. The coating composition as claimed in claim 14, wherein said colouring agent is present in a range of about 0% to about 30% by weight of the composition.

16. The coating composition as claimed in claim 1, further comprising a sweetener selected from the group consisting of sodium saccharin, sucrose, aspartame, neutrame, dextrose, and combinations thereof.

17. The coating composition as claimed in claim 16, wherein said sweetener is present in a range of about 0% to about 10% by weight of the composition.

18. The coating composition as claimed in claim 1, further comprising a flavouring agent.

19. The coating composition as claimed in claim 18, wherein the flavouring agent is present in a range of about 0% to about 15% by weight of the composition.

20. A method for coating substrates such as pharmaceutical tablets, nutritional supplements, food, confectionery, agricultural seeds, and the like, with a film coating composition comprising the steps of:
a) mixing tribasic calcium phosphate at about 2% to about 50% by weight of the composition, a polymer system, and a plasticizer in water or an organic solvent or a water and alcohol solvent to form a smooth dispersion;
b) applying an effective amount of said coating dispersion onto said substrate to form a film on the said substrate, and
c) drying the film coating on said substrate.
